(19) United States
(12) Patent Application Publication
(10) Pub. No.: US 2024/0152258 A1
(43) Pub. Date: May 9, 2024

(54) REAL-TIME SCREEN DATA ENCODING FOR VISUAL AND DIGITAL DECODING

(71) Applicant: Abiomed, Inc., Danvers, MA (US)

(72) Inventor: Muhammad Kamran Sami, Northbridge, MA (US)

(73) Assignee: Abiomed, Inc., Danvers, MA (US)

(21) Appl. No.: 18/388,052

(22) Filed: Nov. 8, 2023

Related U.S. Application Data
(60) Provisional application No. 63/423,659, filed on Nov. 8, 2022.

(51) Int. Cl. G06F 3/0484

(52) U.S. Cl. CPC G06F 3/0484

(57) ABSTRACT

Methods and apparatus for encoding data from a medical device in a visual identifier are described. The method comprises receiving, by the medical device, real-time data corresponding to operation of the medical device, displaying the real-time data on a first portion of a graphical user interface associated with the medical device, encoding at least some of the real-time data in a visual identifier, and displaying on a second portion of the graphical user interface, the two-dimensional visual identifier having the at least some of the real-time data encoded therein, wherein the two-dimensional visual identifier is configured to be scanned by a scanning device. Methods and apparatus for decoding the encoded data are also described.

### CROSS REFERENCE TO RELATED APPLICATIONS

This application claims the benefit under 35 U.S.C. § 119(e) to U.S. Provisional Patent Application No. 63/423,659, filed Nov. 8, 2022, and titled, “REAL-TIME SCREEN DATA ENCODING FOR VISUAL AND DIGITAL DECODING,” the entire contents of which is incorporated herein by reference in its entirety.

### FIELD OF THE INVENTION

This disclosure relates to techniques for real-time encoding of screen data.

### BACKGROUND

Medical devices, when connected to a patient, collect data about the operation of the device and/or a physiological state of the patient. The collected data may be displayed in real-time on a display associated with the medical device. In a typical hospital environment when a clinician needs to record data shown on a display of a medical device, the clinician viewing the display manually enters data into the patient's medical record.

### SUMMARY

Described herein are systems and methods for encoding real-time screen data associated with a medical device for subsequent visual and/or digital decoding.

In one aspect, a method of encoding data from a medical device in a visual identifier is provided. The method comprises receiving, by the medical device, real-time data corresponding to operation of the medical device, displaying the real-time data on a first portion of a graphical user interface associated with the medical device, encoding at least some of the real-time data in a visual identifier, and displaying on a second portion of the graphical user interface, the visual identifier having the at least some of the real-time data encoded therein, wherein the visual identifier is configured to be scanned by a scanning device.

In another aspect, the visual identifier is two-dimensional visual identifier. In another aspect, the two-dimensional visual identifier comprises a barcode. In another aspect, the barcode comprises a quick response (QR) code. In another aspect, the real-time data includes a plurality of hemodynamic parameters. In another aspect, encoding at least some of the real-time data in a visual identifier is performed during operation of the medical device. In another aspect, encoding at least some of the real-time data in a visual identifier is performed at a predetermined time interval. In another aspect, the predetermined time interval is between 1 second and 5 seconds. In another aspect, the method further comprises receiving, via the graphical user interface, a request to generate an updated visual identifier, and encoding at least some of the real-time data in a visual identifier is performed in response to receiving the request.

In another aspect, the method further comprises receiving, via the graphical user interface, a first request to pause encoding of the at least some of the real-time data in the visual identifier, and pausing encoding of the at least some of the real-time data in the visual identifier in response to receiving the first request. In another aspect, the method further comprises receiving, via the graphical user interface, a second request to resume encoding of the at least some of the real-time data in the visual identifier, and resuming encoding of the at least some of the real-time data in the visual identifier in response to receiving the second request.

In another aspect, the method further comprises receiving, via the graphical user interface, a first request to hide the visual identifier on the graphical user interface, and hiding the visual identifier on the graphical user interface in response to receiving the first request. In another aspect, the method further comprises receiving, via the graphical user interface, a second request to show the visual identifier on the graphical user interface, and showing the visual identifier on the graphical user interface in response to receiving the second request.

In another aspect, the method further comprises storing, a plurality of frames of the graphical user interface as a video, and encoding at least some of the real-time data in a visual identifier is performed based, at least in part, on one or more of the plurality of frames of the stored video. In another aspect, the method further comprises displaying, on the graphical user interface, a representation of the video with which a user can interact, and receiving, via the graphical user interface, a selection of a frame of the plurality of frames of the video. In another aspect, encoding at least some of the real-time data in a visual identifier is performed in response to receiving the selection. In another aspect, the medical device is a heart pump. In another aspect, the method further comprises scanning the visual identifier, decoding the real-time data encoded in the visual identifier to produce decoded data, and storing at least some of the decoded data in an electronic health record.

In one aspect, a method of decoding data encoded in a frame of video is provided. The method comprises receiving, by a computing system, video corresponding to screen captures of a medical device display, the video including a plurality of frames, identifying a first frame of the plurality of frames, wherein the first frame includes encoded data, decoding the encoded data in the first frame to generate decoded data, and transmitting the decoded data to a datastore configured to store electronic health records.

In another aspect, the method further comprises extracting, from the first frame, a visual identifier that includes the encoded data, and decoding the encoded data comprises decoding the encoded data from the visual identifier. In another aspect, the visual identifier is a two-dimensional visual identifier. In another aspect, the two-dimensional visual identifier is a barcode or a quick response (QR) code. In another aspect, the method further comprises determining, from the first frame, data corresponding to operation of the medical device, and generating a visual identifier having encoded therein the data corresponding to operation of the medical device, and decoding the encoded data comprises decoding the encoded data from the visual identifier.

In another aspect, the method further comprises scanning with a scanner, at least a portion of the first frame that includes the encoded data to generate scanned data, and decoding the encoded data comprises decoding the scanned data. In another aspect, decoding the scanned data is performed by the scanner. In another aspect, the method further comprises transmitting the scanned data to a computing device, and decoding the scanned data is performed by the computing device. In another aspect, the encoded data includes a plurality of hemodynamic parameters. In another aspect, the medical device display is associated with a heart pump.

In one aspect a data encoding system for a medical device is provided. The data encoding system comprises a controller for the medical device. The controller is configured to receive real-time data from the medical device, display a representation of the real-time data on a first portion of a graphical user interface, encode at least some of the real-time data in a visual identifier, and display, on a second portion of the graphical user interface, the visual identifier including the encoded at least some of the real-time data.

In another aspect, the visual identifier is two-dimensional visual identifier. In another aspect, the two-dimensional visual identifier comprises a barcode. In another aspect, the barcode comprises a quick response (QR) code. In another aspect, the real-time data includes a plurality of hemodynamic parameters. In another aspect, encoding at least some of the real-time data in a visual identifier is performed during operation of the medical device. In another aspect, encoding at least some of the real-time data in a visual identifier is performed at a predetermined time interval. In another aspect, the predetermined time interval is between 1 second and 5 seconds. In another aspect, the controller is further configured to receive, via the graphical user interface, a request to generate an updated visual identifier, and encoding at least some of the real-time data in a visual identifier is performed in response to receiving the request.

In another aspect, the controller is further configured to receive, via the graphical user interface, a first request to pause encoding of the at least some of the real-time data in the visual identifier, and pause encoding of the at least some of the real-time data in the visual identifier in response to receiving the first request. In another aspect, the controller is further configured to receive, via the graphical user interface, a second request to resume encoding of the at least some of the real-time data in the visual identifier, and resume encoding of the at least some of the real-time data in the visual identifier in response to receiving the second request.

In another aspect, the controller is further configured to receive, via the graphical user interface, a first request to hide the visual identifier on the graphical user interface, and hide the visual identifier on the graphical user interface in response to receiving the first request. In another aspect, the controller is further configured to receive, via the graphical user interface, a second request to show the visual identifier on the graphical user interface, and show the visual identifier on the graphical user interface in response to receiving the second request.

In another aspect, the controller is further configured to store, a plurality of frames of the graphical user interface as a video, and encoding at least some of the real-time data in a visual identifier is performed based, at least in part, on one or more of the plurality of frames of the stored video. In another aspect, the controller is further configured to display, on the graphical user interface, a representation of the video with which a user can interact, and receive, via the graphical user interface, a selection of a frame of the plurality of frames of the video. In another aspect, encoding at least some of the real-time data in a visual identifier is performed in response to receiving the selection. In another aspect, the medical device is a heart pump. In another aspect, the controller is further configured to scan the visual identifier, decode the real-time data encoded in the visual identifier to produce decoded data, and store at least some of the decoded data in an electronic health record.

In one aspect, a data decoding system is provided. The data decoding system comprises a video decoder configured to capture a plurality of images of a display of a medical device as a video stream, and separate the video stream into a plurality of frames. The data decoding system further comprises a visual identifier decoder configured to receive a first frame of the plurality of frames, extract a visual identifier from the first frame, decode encoded data from the extracted visual identifier to generate decoded data, the encoded data corresponding to operation of the medical device, and transmit the decoded data to a datastore, the datastore configured to store electronic health records or electronic medical records.

In another aspect, the visual identifier is a two-dimensional visual identifier. In another aspect, the two-dimensional visual identifier is a barcode or a quick response (QR) code. In another aspect, the data decoding system further comprises a scanner configured to scan at least a portion of the first frame that includes the encoded data to generate scanned data, and decoding the encoded data comprises decoding the scanned data. In another aspect, decoding the scanned data is performed by the scanner. In another aspect, the data decoding system further comprises a computing device, the scanner is further configured to transmit the scanned data to the computing device, and decoding the scanned data is performed by the computing device. In another aspect, the encoded data includes a plurality of hemodynamic parameters. In another aspect, the medical device is a heart pump.

### BRIEF DESCRIPTION OF DRAWINGS

FIG. 1 illustrates components of a real-time data encoding/decoding system for a medical device, in accordance with some embodiments of the present technology.

FIG. 2 is a flowchart of a process for encoding real-time data using a two-dimensional visual identifier, in accordance with some embodiments of the present technology.

FIG. 3 is a flowchart of a process for decoding real-time data encoded in one or more frames of video corresponding to screen captures of a display of a medical device, in accordance with some embodiments of the present technology.

### DETAILED DESCRIPTION

The inventor has recognized and appreciated that conventional techniques for recording data shown on a display associated with a medical device may be prone to errors due to visual monitoring of the display and transcribing the observed values onto paper and/or typing the values into an electronic health record. Some embodiments of the present technology relate to improved techniques for capturing data from a display associated with a medical device by encoding the visualized data in a two-dimensional visual identifier (e.g., a barcode), which can be displayed on the screen of the display, and can be scanned by a scanner (e.g., a commercially available barcode scanner) and/or can be decoded by a digital decoder. As described herein, the data encoded in the two-dimensional visual identifier can be periodically updated (e.g., every 5 seconds) in real-time as the medical device is in operation, such that the identifier includes encoded data that represents a current (or relatively current) state of the data associated with operation of the medical device. Automating the capture and encoding of real-time data corresponding to operation of a medical device may reduce errors caused by manual capture and entry of such information, as required by some conventional medical devices.

As described herein, medical devices are typically configured to permit only static data capture and do not provide the ability to capture data shown at any given time on the screen of the device. In some embodiments, encoding data within a two-dimensional visual identifier (e.g., a barcode, QR code) may be performed in real-time and made available to a clinician for scanning via a hand-held scanner. Once scanned, the encoded data can be decoded and stored in an electronic health record (EHR) or electronic medical record (EMR) database.

In some embodiments, a video corresponding to screen captures of the display of the medical device may be decoded and split into individual frames. The portion of the display where the two-dimensional visual identifier is located may be decoded using a decoder and the decoded data may be converted to a digital format that can be stored directly into the EHR/EMR database.

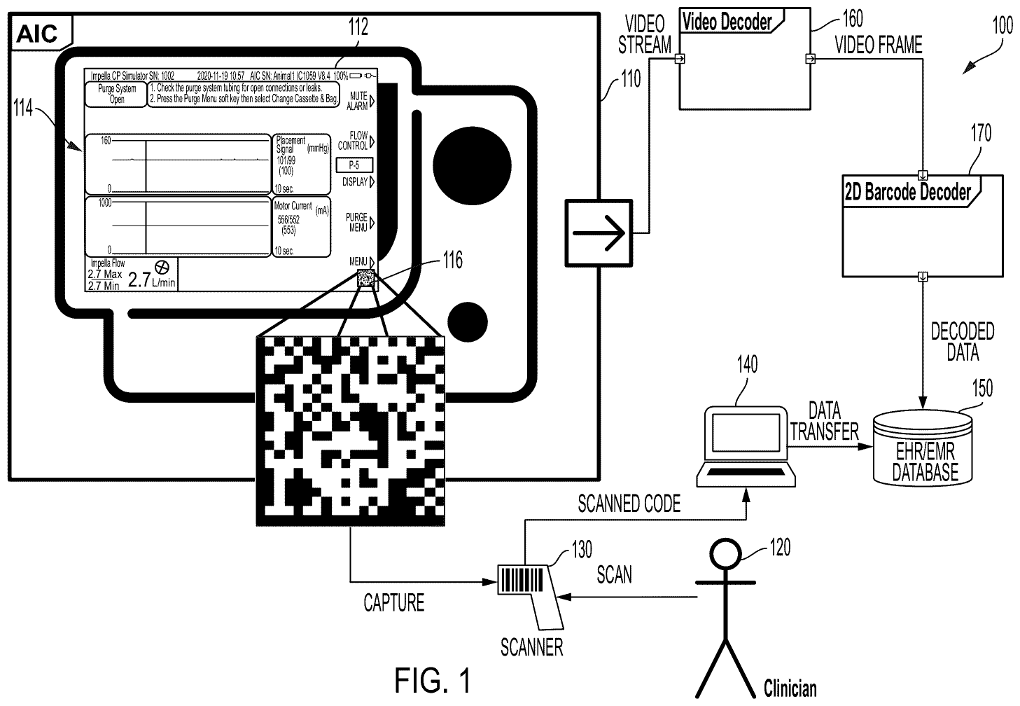

FIG. 1 illustrates a system 100 for dynamically capturing and encoding real-time data from a medical device in accordance with some embodiments of the present disclosure. System 100 may include medical device 110 configured to display data associated with operation of the medical device. In the example shown in FIG. 1, medical device 110 is a controller for a heart pump system that includes the controller and a heart pump (not shown) configured to be inserted into a patient's heart. The controller is configured to receive information from the heart pump corresponding to operation of the heart pump system. For instance, the received information may include a plurality of hemodynamic parameters, such as blood flow (Q) through the pump, aortic pressure (AOP), left ventricular pressure (LVP), left ventricular end diastolic pressure (LVEDP), continuous cardiac output (CCO), etc. Although medical device 110 is shown in FIG. 1 as a portion of a system (i.e., a controller) that includes another device (e.g., a heart pump), it should be appreciated that medical device 110 may, in some instances, be implemented as a single device that both measures information associated with a patient and/or operation of the medical device and processes that information for display.

The medical device 110 (e.g., a controller) may include a display 112 configured to show a representation of at least some of the data received from another device (e.g., a heart pump), with the data corresponding to operation of the medical device. For instance, display 112 may be configured to show one or more numbers, graphs, charts or other representations of the data. The data may be received by medical device 110 in real-time (e.g., as the data is being sensed, or with a short delay after the data is sensed) to enable the display 112 to be updated with current data to enable a clinician or other user to understand a current operating state of the medical device by viewing display 112. In such instances, the data may be referred to as “real-time data.” As shown in FIG. 1, a representation of the received real-time data may be displayed on a first portion of a graphical user interface (GUI) shown on display 112. A user may interact with the GUI to change the data that is displayed on the GUI and/or to control one or more operations of the medical device. For instance, in the example of a controller that controls a heart pump, the user may interact with the GUI to view historical data for one or more measured data metrics and/or to change a pump speed of the heart pump. In some embodiments, medical device 110 may include a communications component configured to transmit at least some of the received data to a remote computing device (e.g., a smartphone, a tablet computer, a laptop computer) over one or more networks. The remote computing device may include display 112 configured to show at least some of the received data. The functionality provided by a GUI displayed on the remote computing device may be the same or different from a GUI shown on a display integrated with the medical device 110. For instance, a user may be able to interact with a GUI shown on an integrated display to control operation of a connected device (e.g., a heart pump), whereas a user viewing a GUI shown on a remote computing device display may only be able to view the data displayed thereon, but may not be permitted to control operation of the connected device to, for example, ensure that only authorized users are able to change operation of the connected device via the remote computing device.

In addition to showing a representation of at least some of the received data on a first portion of the GUI, medical device 110 may be configured to show, in a second portion of the GUI, a two-dimensional (2D) visual identifier 116. The 2D visual identifier may be a barcode or any other suitable 2D visual identifier. When implemented as a barcode, the 2D visual identifier may have any suitable format, examples of which include, but are not limited to a dot matrix format (as shown in FIG. 1) and a quick response (QR) code format. By providing the 2D visual identifier on the screen, a conventional handheld scanner device (e.g., a barcode reader and/or a smartphone) may be used to scan the 2D visual identifier to retrieve information encoded therein. For example, as shown in FIG. 1, a clinician 120 may use scanner 130 (e.g., a barcode scanner) to scan the 2D visual identifier 116 shown on the display 112. As will be appreciated, in some embodiments, the scanner may include a mobile device (e.g., a mobile phone).

In accordance with some embodiments, the 2D visual identifier may be encoded with at least some of the received real-time data corresponding to operation of the medical device. In some embodiments, all of the received real-time data may be encoded in the 2D visual identifier. In other embodiments, only a subset of the received real-time data may be encoded in the 2D visual identifier. The subset of data to encode may be selectable by a clinician or other user by, for example, interacting with the GUI shown on display 112. In some embodiments, at least some of the real-time data may be processed prior to being encoded in the 2D visual identifier. For instance, one or more received values received over a period of time may be processed to determine an average, maximum, minimum, or some other suitable metric over the period of time, and the processed data may be encoded in the 2D visual identifier.

In accordance with some embodiments, the data encoded in the 2D visual identifier 116 may be updated dynamically during operation of the medical device, such that the encoded data associated with the 2D visual identifier represents current or near current data corresponding to the operation of the medical device. For instance, the encoded data may be updated at a predetermined time interval. As an example, at least some of the received real-time data may be encoded every time the GUI is updated on display 112 (e.g., at a frame rate of 10 Hz). In such an instance, the 2D visual identifier may be updated for each updated frame of the display. In other embodiments, the data encoded in the 2D visual identifier may be updated less frequently than every frame. For instance, it may be beneficial to update the 2D visual identifier less frequently than every frame update of display 112 to ensure that the encoded data remains constant for at least the amount of time needed to scan the 2D visual identifier using scanner 130. Again, in such embodiments, the encoded data may be updated at a prescribed time interval. For example, in such embodiments, the encoded data may be updated every 1 second, every 2 seconds, every 5 seconds, every 10 seconds, or longer.

In some embodiments, the encoded data associated with 2D visual identifier 116 may be updated “on-demand” in response to a user input to update the 2D visual identifier rather than being updated automatically after a predetermined amount of time has elapsed. For instance, clinician 120 may interact with the GUI shown on display 112 to instruct medical device 110 to generate a 2D visual identifier encoded with current data. In response to receiving the request, medical device 110 may be configured to update the encoded data in 2D visual identifier 116 (e.g., by generating a new 2D visual identifier to replace the previous 2D visual identifier shown on the display 112).

In some embodiments, the GUI may be configured to enable a user (e.g., a clinician) to pause or resume encoding of real-time data into the 2D visual identifier. For instance, the GUI may include a button that enables the user to pause encoding the data. Pausing encoding of the data may, for example, provide sufficient time for the user to scan the 2D visual identifier 116 with scanner 130. The user may then interact with GUI to resume encoding of the data in 2D visual identifier, for example, after the 2D visual identifier has been scanned. By enabling the user to pause encoding of the data in 2D visual identifier 116, the system may provide real-time updates of the data encoded in 2D visual identifier 116, while also keeping the data encoded in 2D visual identifier 116 constant, when desired (such as while scanning).

The inventor has recognized that the user may not want the 2D visual identifier 116 to be shown on the GUI at all times. For instance, the representation of the 2D visual identifier 116 may need to be large enough to be scanned by scanner 130, and as such may take up valuable real estate on the GUI that could be used to display other information when scanning is not desired. Accordingly, in some embodiments, the GUI shown on display 112 may be configured to enable a user to hide or show the 2D visual identifier 116. For instance, the user may interact with a button displayed on the GUI to hide or show the 2D visual identifier 116. In such embodiments, the encoded data in the 2D visual identifier 116 may be updated even when the 2D visual identifier 116 is hidden (i.e., not shown on the GUI). In other embodiments, the encoded data in the 2D visual identifier 116 may not be updated when the 2D visual identifier 116 is hidden, but may instead be updated in response to a user request that the 2D visual identifier 116 be shown on the GUI. In some embodiments, when the user requests that the 2D visual identifier 116 be shown on the GUI, the 2D visual identifier 116 may be shown in the same location of the GUI. In other embodiments, the 2D visual identifier 116 may be shown in a different location of the GUI when a request to show the 2D visual identifier is received. By showing the 2D visual identifier 116 in a different location on the GUI, certain important data currently shown on the GUI may not be covered with the 2D visual identifier 116 when shown. Additionally, by allowing the 2D visual identifier 116 to appear in different places on the GUI, the screen real estate on the GUI where the 2D visual identifier 116 last appeared can be repurposed for displaying information when the 2D visual identifier is hidden.

The 2D visual identifier 116 is shown in FIG. 1 as a square barcode. However, it should be appreciated that the 2D visual identifier 116 may be represented using other formats and/or shapes (e.g., rectangles, circles, polygons, etc.). In some embodiments, the 2D visual identifier 116 may be represented as black and white pixels arranged in a particular shape (e.g., a square), wherein the encoded data is encoded based on the pattern of black/white pixels within the shape. In some embodiments, the 2D visual identifier 116 may be rendered in grayscale and/or color and at least some of the encoded data or additional data may be encoded using the grayscale and/or color space dimensions. For instance, in some embodiments that use a 2D visual identifier 116 rendered in color, the color space of the 2D visual identifier may be used to embed data for redundancy and/or error correction. In some embodiments, the color space may be used to extend the payload for encoding data into the 2D visual identifier, such that additional data may be encoded if the color space was not used.

As also shown in FIG. 1, a clinician 120 (or other user) may use scanner 130 to scan the 2D visual identifier 116. The scanner 130 may decode the encoded data in the 2D visual identifier and send the decoded data to computing system 140 as a scanned code. The computing system may process the decoded data and store the processed data into EHR/EMR database 150. In some embodiments, the scanner 130 may not decode the encoded data in the 2D visual identifier, but instead may pass the encoded data to computing system 140, which may be configured to decode the encoded data. The decoded data may then be sent to EHR/EMR database 150. In some embodiments, the encoded data may not be decoded by the scanner 130 or the computing system 140, but may be stored in EHR/EMR database 150 in encoded form. The encoded data may then be decoded from the patient's EHR/EMR at a later point in time. By providing a system in which data corresponding to operation of the medical device can be automatically captured and extracted from the medical device, and then transferred into a patient's EHR/EMR, the process of manually writing and/or typing such values, required for some conventional medical device systems, can be minimized or altogether avoided.

As will be appreciated, although the scanner is described as decoding the encoded data in the visual identifier in this embodiment, it will be appreciated that in other embodiments, the scanned code may be sent to the computing system, with the computing system configured to decode the scanned code. In such embodiments, the decoded data may again be sent from the computing system to the EHR/EMR database.

Additionally or alternatively to enabling a clinician 120 or other user to physically scan a visually presented and dynamically updated 2D visual identifier to capture data associated with operation of a medical device, in some embodiments, screen captures of the GUI shown on display 112 may be stored as a plurality of frames of a video. As shown in FIG. 1, the series of time-based screen captures of the GUI may be stored as a video stream, which may be captured by a video decoder 160. Video decoder 160 may be configured to capture the video from the medical device in real-time and separate the video into a plurality of frames. In some embodiments, video decoder 160 may include, or be in communication with, a display configured to present the plurality of frames to a user, such as a clinician. The user can then review the historical record of the operating state of the medical device captured in the frames of video to identify a frame that includes information of interest for recording into a patient's EHR/EMR. Upon selection of a particular video frame, that video frame may be sent from video decoder 160 to visual identifier decoder 170, which may extract the 2D visual identifier from the selected video frame and decode the encoded data in the 2D visual identifier. For instance, visual identifier decoder 170 may be configured to decode the received video frame and extract the encoded data in the 2D visual identifier. The decoded data may then be sent to the ERR/EMIR database 150.

Although a user is described herein as performing the frame selection, in some embodiments, selection of a frame of video from the video stream may be performed automatically (e.g., without user selection). For instance, the video decoder 160 or some other component communicatively coupled with the video decoder 160 may analyze the frames of video to identify one or more frames associated with real-time data from the medical device that are outside of specified bounds (e.g., arterial pressure was too low or too high relative to prescribed threshold values). In response to detecting a frame of video associated with such “abnormal” real-time data, the frame may be automatically selected and sent to visual identifier decoder 170 for extraction of the 2D visual identifier as described herein.

In yet further embodiments, a combination of automated and manual processes may be used to identify a frame of video of interest. For example, an automated process may identify candidate frames of interest based on the detection of associated abnormal real-time data, and the candidate frames may be presented to a user for review and selection. By presenting candidate frames of interest to the user, the burden on the user to review every frame in the video stream may be reduced.

In embodiments in which scanning of a displayed 2D visual identifier is not required (e.g., when a video stream of the screen capture images from the display 112 is analyzed), the real-time data corresponding to operation of the medical device may be embedded within the image shown on display 112 in a manner than is not discernable to the human eye. For instance, the image for a frame may be embedded with hints provided within the image that are not discernable to the human eye, but which may identify the encoded real-time data, which can be used to subsequently decode the data for entry into a patient's EHR/EMR.

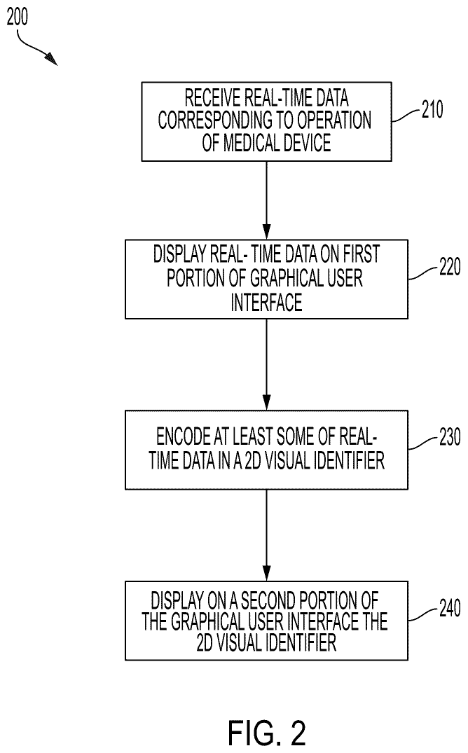

FIG. 2 illustrates a process 200 for encoding real-time data from a medical device in a two-dimensional visual identifier shown on a display associated the medical device, in accordance with some embodiments. Process 200 begins in act 210, where real-time data corresponding to operation of a medical device may be received. For instance, one or more parameters describing physiological characteristics of a patient associated with the medical device and/or one or more values associated with operation of the medical device (e.g., pump speed for a heart pump) may be received during operation of the medical device. Process 200 then proceeds to act 220, where the received real-time data may be displayed on a first portion of a graphical user interface (GUI) shown on a display associated with the medical device. Process 200 then proceeds to act 230, where at least some of the received real-time data may be encoded in a two-dimensional (2D) visual identifier. Process 200 then proceeds to act 240, where the 2D visual identifier may be displayed on a second portion of the GUI. As described herein, the 2D visual identifier can then be scanned by a clinician or another user using a scanner (e.g., a barcode scanner), which captures the 2D visual identifier and decodes the encoded data, which may be stored in a patient's EHR/EMR.

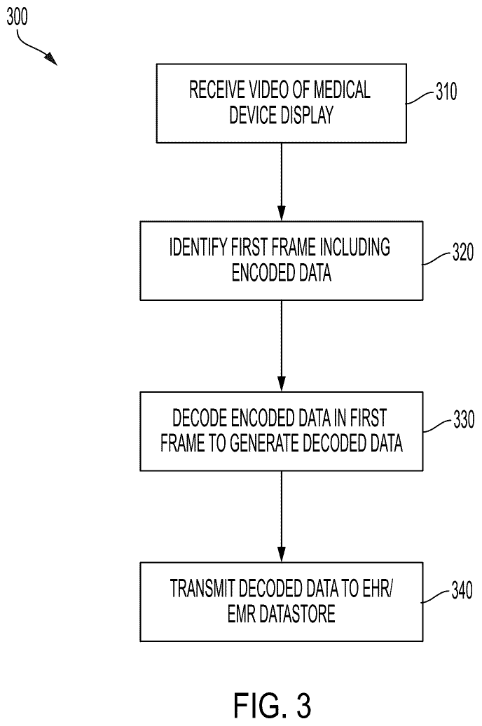

FIG. 3 illustrates a process 300 for decoding encoded data stored in video of a medical device display, in accordance with some embodiments. Process 300 begins in act 310, in which video of a medical device display may be received. As described herein, a video decoder may capture screen images from the medical device display as a video stream and separate the video stream into a plurality of frames that can be further analyzed. Process 300 then continues to act 320, where a first frame of the plurality of frames including encoded real-time data may be identified. As described herein, the first frame may be selected manually, automatically, or using a combination of automated and manual processes. Process 300 then proceeds to act 330, where the encoded data in the first frame may be decoded to generate decoded data. As described herein, in some embodiments, decoding the encoding data includes decoding the first frame, extracting a 2D visual identifier from the decoded frame, and decoding the encoded data from the extracted 2D visual identifier. Process 300 then proceeds to act 340, where the decoded data may be transmitted to an electronic health record (EHR) or electronic medical record (EMR) datastore configured to store an ERR/EMIR for a patient associated with the medical device.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will also recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method of encoding data from a medical device in a visual identifier, the method comprising:
receiving, by the medical device, real-time data corresponding to operation of the medical device;
displaying the real-time data on a first portion of a graphical user interface associated with the medical device;
encoding at least some of the real-time data in a visual identifier; and
displaying on a second portion of the graphical user interface, the visual identifier having the at least some of the real-time data encoded therein, wherein the visual identifier is configured to be scanned by a scanning device.

2. The method of claim 1, wherein the visual identifier is a two-dimensional visual identifier.

3. The method of claim 2, wherein the two-dimensional visual identifier comprises a barcode.

4. The method of claim 3, wherein the barcode comprises a quick response (QR) code.

5. The method of claim 1, wherein the real-time data includes a plurality of hemodynamic parameters.

6. The method of claim 1, wherein encoding at least some of the real-time data in a visual identifier is performed during operation of the medical device.

7. The method of claim 6, wherein encoding at least some of the real-time data in a visual identifier is performed at a predetermined time interval.

8. The method of claim 7, wherein the predetermined time interval is between 1 second and 5 seconds.

9. The method of claim 6, further comprising:
receiving, via the graphical user interface, a request to generate an updated visual identifier,
wherein encoding at least some of the real-time data in a visual identifier is performed in response to receiving the request.

10. The method of claim 1, further comprising:
receiving, via the graphical user interface, a first request to pause encoding of the at least some of the real-time data in the visual identifier; and
pausing encoding of the at least some of the real-time data in the visual identifier in response to receiving the first request.

11. The method of claim 10, further comprising:
receiving, via the graphical user interface, a second request to resume encoding of the at least some of the real-time data in the visual identifier; and
resuming encoding of the at least some of the real-time data in the visual identifier in response to receiving the second request.

12. The method of claim 1, further comprising:
receiving, via the graphical user interface, a first request to hide the visual identifier on the graphical user interface; and
hiding the visual identifier on the graphical user interface in response to receiving the first request.

13. The method of claim 12, further comprising:
receiving, via the graphical user interface, a second request to show the visual identifier on the graphical user interface; and
showing the visual identifier on the graphical user interface in response to receiving the second request.

14. The method of claim 1, further comprising:
storing, a plurality of frames of the graphical user interface as a video,
wherein encoding at least some of the real-time data in a visual identifier is performed based, at least in part, on one or more of the plurality of frames of the stored video.

15. The method of claim 14, further comprising:
displaying, on the graphical user interface, a representation of the video with which a user can interact; and
receiving, via the graphical user interface, a selection of a frame of the plurality of frames of the video.

16. The method of claim 15, wherein encoding at least some of the real-time data in a visual identifier is performed in response to receiving the selection.

17. The method of claim 1, wherein the medical device is a heart pump.

18. The method of claim 1, further comprising:
scanning the visual identifier;
decoding the real-time data encoded in the visual identifier to produce decoded data; and
storing at least some of the decoded data in an electronic health record.

19-28. (canceled)

29. A data encoding system for a medical device, the data encoding system comprising:
a controller for the medical device, the controller configured to:
receive real-time data from the medical device;
display a representation of the real-time data on a first portion of a graphical user interface;
encode at least some of the real-time data in a visual identifier; and
display, on a second portion of the graphical user interface, the visual identifier including the encoded at least some of the real-time data.

30-46. (canceled)

47. A data decoding system, comprising:
a video decoder configured to:
capture a plurality of images of a display of a medical device as a video stream; and
separate the video stream into a plurality of frames; and
a visual identifier decoder configured to:
receive a first frame of the plurality of frames;
extract a visual identifier from the first frame;
decode encoded data from the extracted visual identifier to generate decoded data,
the encoded data corresponding to operation of the medical device; and
transmit the decoded data to a datastore, the datastore configured to store electronic health records or electronic medical records.

48-54. (canceled)
